(19) United States
(12) Patent Application Publication
(10) Pub. No.: US 2012/0152243 A1
(43) Pub. Date: Jun. 21, 2012

(54) Breathing Apparatus

(75) Inventors: Richard Hingley, Hythe (GB); Eric Pagan, Hythe (GB); Peter Ryan Smith, Canterbury (GB)

(73) Assignee: Smiths Medical International Limited, Kent (GB)

(21) Appl. No.: 13/392,679

(22) PCT Filed: Aug. 31, 2010
(86) PCT No.: PCT/GB10/01637

§ 371 Date: Feb. 27, 2012

(30) Foreign Application Priority Data
Sep. 25, 2009 (GB) 0916845.1

(51) Int. Cl. A61M 16/12; A63B 21/00; A63B 31/00; A63B 22/00; A63B 22/06

(52) U.S. Cl. 128/203.12; 482/51; 482/57; 482/55; 482/92

(57) ABSTRACT

A tent-like structure has a compact configuration and can be expanded to an expanded configuration large enough totally to enclose a patient. A recirculating gas supply arrangement supplies a mixture of oxygen and helium to the inside of the structure. The structure may contain exercise equipment, such as a static cycle, so that the patient can exercise in the gas mixture. The structure could also be used over a swimming pool. A weight or other means at the lower end of the structure helps to seal it with the floor surface and reduce escape of gas.

This invention relates to breathing apparatus.

The invention is more particularly concerned with apparatus for providing breathing gas to patients having respiratory problems.

It is known that patients with respiratory problems can benefit from a gas mixture of oxygen and helium, the latter gas reducing the patient's work of breathing. Helium, however, is relatively scarce and expensive so conventional ventilation techniques where exhaled gas is exhausted to atmosphere are very wasteful and costly. The high cost is such that treatment with helium can only usually be provided to patients with severe respiratory problems.

Various ways are used to supply breathing gas to a patient, such as face masks, nasal masks or mouthpieces. These can be uncomfortable in prolonged use, often causing damage to the skin around the mouth or nose; they usually prevent the patient talking and often make it difficult to wear spectacles. Alternatively, a hood may be used to enclose the entire head. None of these arrangements are very comfortable to use during exercise or similar therapy, which can be important in aiding the patient's recovery.

It is an object of the present invention to provide alternative breathing apparatus.

According to one aspect of the present invention there is provided breathing apparatus, characterised in that the apparatus includes a tent-like structure arranged to be expansible from a relatively compact configuration to an expanded configuration for enclosing substantially the entire of a patient, and that the apparatus includes a gas supply arrangement for supplying breathing gas to the interior of the tent-like structure when in an expanded configuration.

The breathing apparatus preferably includes exercise equipment arranged to be enclosed by the tent-like structure when the structure is in an expanded configuration. The exercise equipment may include at least one of the following: a cycle, an item of aerobic exercise equipment, an item of resistance exercise equipment and a swimming pool. The gas supply arrangement may include a recirculating or rebreathing system and is preferably arranged to supply a breathing gas mixture including helium. The tent-like structure preferably includes means to hold a lower edge of the structure in sealing contact with a floor. The means for holding a lower edge of the structure in sealing contact with the floor may include at least one of the following: a weight, magnetic means, an adhesive and a hook and loop fastener arrangement. The tent-like structure preferably has a closable door by which the user can enter and leave the apparatus. The structure may have an inner and outer door forming an airlock for entry to or exit from the structure. The gas supply arrangement may be arranged to reduce the supply of a gas to the structure towards the end of an exercise period in order to reduce loss of supplied gas when the user leaves the structure. The structure may be foldable from a compact to an expanded configuration and includes a plurality of support members that are moved away from one another when the structure is expanded. The tent-like structure may be inflatable to the expanded configuration. The apparatus may include a trolley supporting the gas supply arrangement and the tent-like structure. The tent-like structure may be supported from an elevated support and be lowered over a patient.

Apparatus according to the present invention will now be described, by way of example, with reference to the accompanying drawings, in which:

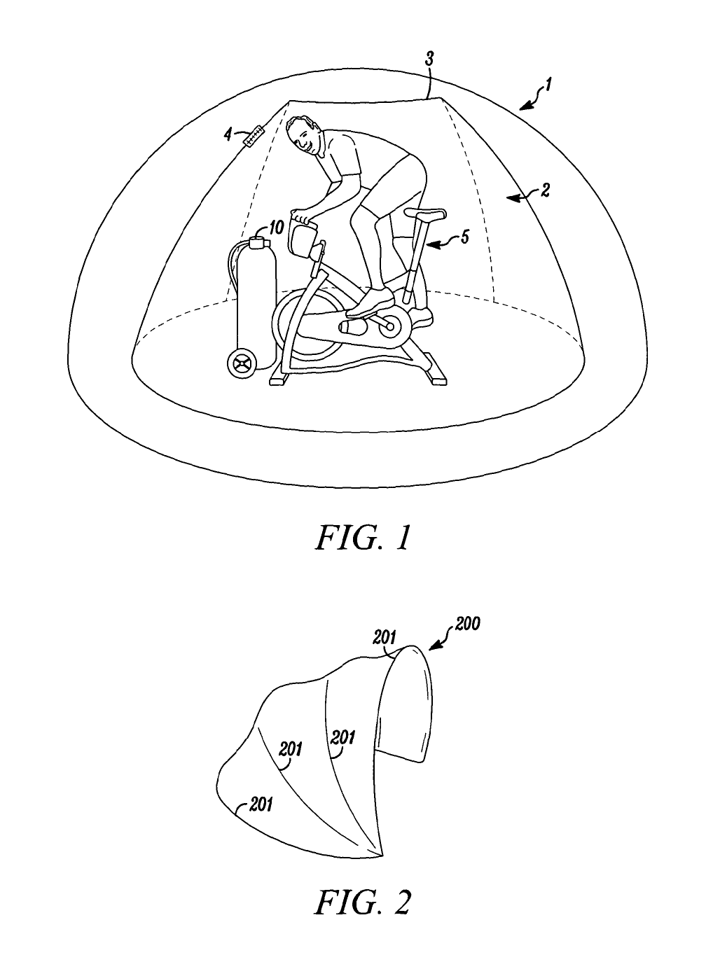

FIG. 1 is a cut-away side elevation view of an inflated tent;

FIG. 2 is a perspective view of a foldable corrugated tent in a partly erected state;

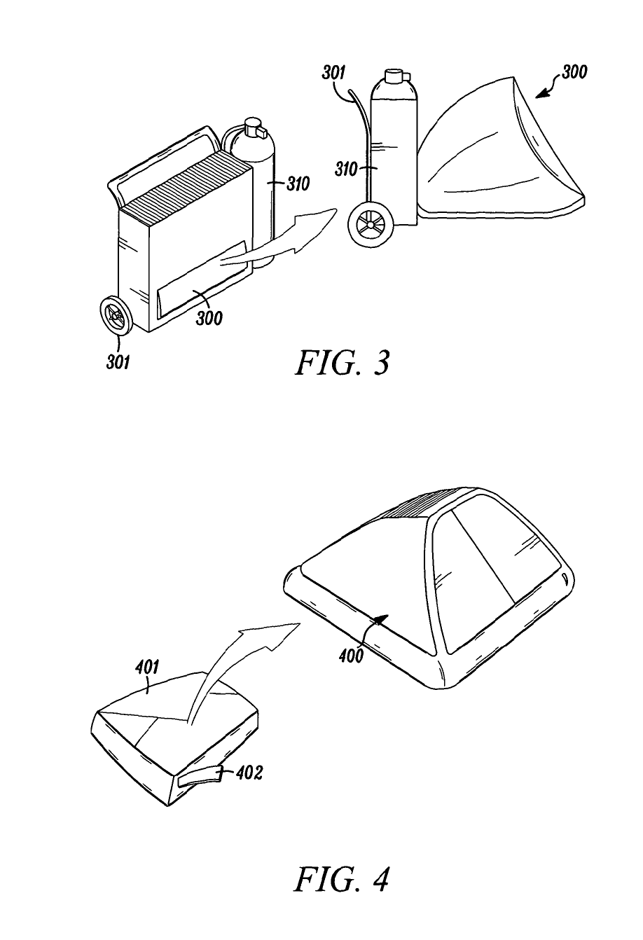

FIG. 3 shows a tent incorporated with a trolley;

FIG. 4 shows an inflatable tent in a bag; and

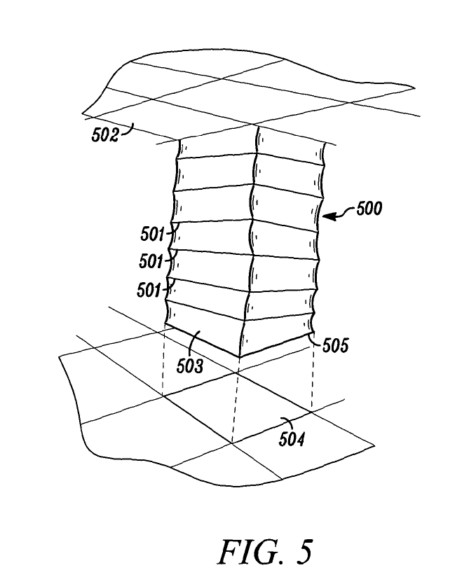

FIG. 5 shows a tent of concertina construction suspended from a ceiling.

With reference first to FIG. 1 there is shown an inflated tent structure or enclosure 1 of hemispherical, domed shape made of a lightweight, flexible, gas-impermeable material such as a plastics fabric or non-woven. The structure 1 is about 3 m high in the centre with a diameter of about 6 m. A generally triangular shape door 2 is attached to the structure along a flexible hinge 3 at its upper edge and is closed and opened by means of a zip fastener 4, Velcro or the like. The patient enters the structure by opening the door 2 and then sealing it closed behind him. The structure encloses an item of conventional exercise equipment, such as a static cycle 5, as shown, or any other conventional aerobic or resistance exercise equipment. Also, the apparatus is shown as containing a gas supply 10 although it will be appreciated that the gas supply could be located outside the tent structure and connected to it by suitable tubing or ducting. The gas supply 10 preferably, although not essentially, takes the form of a heliox rebreather supply arranged to provide a helium and oxygen mixture within the tent structure 1 by recirculating gas from the tent. More particularly, the supply 10 includes canisters of helium and oxygen gas, a filter and a scrubber (none shown) for removing unwanted substances and carbon dioxide, and some form of gas flow means, such as a pump (again not shown). The supply 10 creates a positive pressure inside the tent 1 of breathing gas so that the patient can breath freely while exercising on the equipment 5 without the need for a face mask, hood or other fitted breathing interface. The patient can rebreath their exhaled gases. The heavier carbon dioxide may sink to the lower part of the enclosure 1 so it would be preferable for the gas extraction inlet of the gas supply 10 to be located in this region. The extracted gas is filtered and scrubbed, with fresh helium and oxygen being added as necessary before it is returned to the inside of the tent 1. As the fresh gas enters the tent 1 the lighter helium may help displace the older gas downwards to the gas extraction inlet for removal. This arrangement enables a part at least of the expensive helium gas to be captured for reuse. The apparatus could be arranged to stop supplying helium to the interior of the tent towards the end of the exercise period, to reduce the amount of helium that will be lost when the patient opens the door 2 to leave the enclosure 1. An alternative, or additional arrangement for minimizing gas loss would be for the enclosure to have an inner and outer door arrangement to form an air lock so that gas does not escape directly from the interior of the enclosure to atmosphere when the patient enters or exits the enclosure.

In an alternative arrangement the tent structure could have a double-layer wall with a cavity between the layers. In such an arrangement the cavity would be used as a passage for extraction or supply of gases. The tent structure could include two or more items of exercise equipment, such as for simultaneous use by several patients or for enabling a patient to perform different exercises in the same environment.

Alternatively, the structure could be positioned over a swimming pool to enable the patient to swim. The tent could cover the entire of a small pool, perhaps a pool provided with a flowing stream of water against which the patient would swim, or the tent structure could be arranged to float on the surface of a larger pool and to be moved with the swimming patient. Contact of the lower end of the tent structure with the water would provide a very effective gas seal. In the context of a swimming pool, the water surface provides the equivalent of a floor.

FIG. 2 shows an alternative tent structure 200 that is of a foldable, corrugated style with several support members or stiffening U-shape hoops 201 that can be folded together to form a substantially flat configuration, or folded out to through 180° by separating the hoops to provide a complete dome.

FIG. 3 illustrates an arrangement where both the gas supply 310 and tent structure 300 are mounted on a trolley 301 so that it can be moved to any desired location for erection.

FIG. 4 shows an inflatable tent structure 400 compacted within a bag 401 and provided with a release cord 402 similar to inflatable rafts. Such an arrangement would require a minimum space for storage and would be suitable for emergency use, such as treating victims of toxic gas or smoke inhalation at the scene of an accident or fire. The tent structure 400 could be compacted for storage after use by using the rebreather unit pump to withdraw gas from the inflated parts of the structure.

The tent structure, could include an integral floor further to minimise gas leakage.

FIG. 5 shows a tent structure 500 of square or rectangular section and of concertina or Chinese lantern configuration with parallel stiffening frames or support members 501. The structure 500 is suspended at its upper end from a ceiling 502 or other elevated support so that it can be tied or otherwise retained in a retracted, elevated position when not in use. To use the tent structure 500 it is lowered over a piece of exercise equipment and the patient (neither shown) with the lower end 503 of the structure resting on the floor 504. To improve the gas seal with the floor 504 the tent 500 preferably includes some means to hold its lower end 503 in sealing contact with the floor. The lower rim 505 of tent 500 could be weighted to help it sit on the floor 504, or it could include a magnetic member that interacts with a metal plate or the like on the floor to provide an attractive force. Alternatively, the lower end of the tent could be adhesive or have a Velcro hook fabric arranged to attach with loops of a fibre material, such as a carpet on the floor surface. It will be appreciated that there are various other arrangements that could be used to enhance the seal between the tent and the floor.

1-14. (canceled)

15. Breathing apparatus including, a tent-like structure arranged to be expansible from a relatively compact configuration to an expanded configuration for enclosing substantially the entire of a patient, characterized in and that the apparatus includes a gas supply arrangement for supplying a breathing gas mixture including helium to the interior of the tent-like structure when in an expanded configuration.

16. Breathing apparatus according to claim 15, characterized in that the apparatus includes exercise equipment arranged to be enclosed within the tent-like structure when the structure is in an expanded configuration.

17. Breathing apparatus according to claim 16, characterized in that the exercise equipment includes at least one of the following: a cycle, an item of aerobic exercise equipment, an item of resistance exercise equipment and a swimming pool.

18. Breathing apparatus according to claim 15, characterized in that the gas supply arrangement includes a recirculating or rebreathing system.

19. Breathing apparatus according to claim 15, characterized in that the tent-like structure includes an arrangement for holding a lower edge of the structure in sealing contact with a floor.

20. Breathing apparatus according to claim 19, characterized in that the arrangement for holding a lower edge of the structure in sealing contact with a floor includes at least one of the following: a weight, magnetic means, an adhesive and a hook and loop fastener arrangement.

21. Breathing apparatus according to claim 15, characterized in that the tent-like structure has a closable door by which the user can enter and leave the apparatus.

22. Breathing apparatus according to claim 21, characterized in that the structure has an inner and outer door forming an airlock for entry to or exit from the structure.

23. Breathing apparatus according to claim 15, characterized in that the gas supply arrangement is arranged to reduce the supply of a gas to the structure towards the end of an exercise period in order to reduce loss of supplied gas when the user leaves the structure.

24. Breathing apparatus according to claim 15, characterized in that the structure is foldable from a compact to an expanded configuration, and that the structure includes a plurality of support members that are moved away from one another when the structure is expanded.

25. Breathing apparatus according to claim 15, characterized in that the tent-like structure is inflatable to the expanded configuration.

26. Breathing apparatus according to claim 15, characterized in that the apparatus includes a trolley supporting the gas supply arrangement and the tent-like structure.

27. Breathing apparatus according to claim 15, characterized in that the tent-like structure is supported from an elevated support and can be lowered over the patient.
